(19) United States
(12) Patent Application Publication
(10) Pub. No.: US 2015/0191789 A1
(43) Pub. Date: Jul. 9, 2015

(54) METHODS AND MATERIALS FOR IDENTIFYING MAMMALS HAVING PROSTATE CANCER

(71) Applicant: MAYO FOUNDATION FOR MEDICAL EDUCATION AND RESEARCH, Rochester, MN (US)

(72) Inventors: Farhad Kosari, Ellsworth, WI (US); John C. Cheville, Pine Island, MN (US); R. Jeffrey Karnes, Rochester, MN (US); George Vasmatzis, Oronoco, MN (US)

(73) Assignee: MAYO FOUNDATION FOR MEDICAL EDUCATION AND RESEARCH, Rochester, MN (US)

(21) Appl. No.: 14/377,604

(22) PCT Filed: Feb. 6, 2013
(86) PCT No.: PCT/US13/24958

§ 371 Date: Aug. 8, 2014

Related U.S. Application Data
(60) Provisional application No. 61/597,623, filed on Feb. 10, 2012.

(51) Int. Cl. C12Q 1/68

(52) U.S. Cl. CPC C12Q 1/6886; C12Q 2600/158

(57) ABSTRACT

This document provides methods and materials related to identifying prostate cancer in male mammals. For example, methods and materials for assessing a benign prostate sample (e.g., benign prostate tissue or cells) to determine whether or not a mammal has prostate cancer are provided.

### CROSS-REFERENCE TO RELATED APPLICATIONS

This application claims priority to U.S. Provisional Application Ser. No. 61/597,623, filed on Feb. 10, 2012. The disclosure of the prior application is considered part of (and is incorporated by reference in) the disclosure of this application.

### BACKGROUND

1. Technical Field

This document relates to methods and materials involved in identifying prostate cancer in male mammals. For example, this document provides methods and materials for assessing benign prostate tissue to determine whether or not a mammal has prostate cancer.

2. Background Information

The diagnosis of prostate cancer (PCa) is based mainly on needle biopsy evaluation of the prostate gland. However, the needle biopsy procedure can have a 30% false negative rate due to sampling error (Patel et al., Urology, 63:87-89 (2004) and Stewart et al., J. Urol., 166:86-92 (2001)). As a result, many of the approximately 800,000 men found with a negative biopsy in the United States each year undergo repeat biopsies, which can be frustrating for both patients and urologists. For benign prostate needle biopsy specimens that lack atypical small acinar proliferation (ASAP) or high-grade prostatic intraepithelial neoplasia and cancer, there is no additional information that can be gained by pathologic assessment. However, the potential that these prostate glands have incurred the initial neoplastic transformations or harbor prostate cancer is significant. Despite a lack of morphologic changes, there is a considerable body of evidence suggesting that molecular alterations associated with tumor in adjacent non-neoplastic cells, the so called “tumor field effect,” can provide valuable clues regarding the presence of tumor. The prostatic tumor field effect was first reported more than 10 years ago based on subtle histological changes in the tissue architecture and cytology in benign tissue adjacent to and at some distance from PCa (Montironi et al., J. Pathol., 182:442-449 (1997)). Subsequent studies have documented tumor-associated molecular alterations in non-neoplastic tissue adjacent to PCa in resected specimens, and notably in needle biopsy specimens (Dhir et al., J. Urol., 171:1419-1423 (2004) and Troyer et al., Cancer Epidemiol. Biomarkers Prey., 18:2717-2722 (2009)). Several investigators used microarrays to identify expression alterations associated with PCa field effects (Chandran et al., BMC Cancer, 5:45 (2005); Haaland et al., Int. J. Oncol., 35:537-546 (2009); and Risk et al., Clin. Cancer Res., 16:5414-5423 (2010)). These profiling studies were often independent of PCa and appeared to include limited or no independent validation.

### SUMMARY

This document provides methods and materials related to identifying prostate cancer in male mammals. For example, this document provides methods and materials for assessing a benign prostate sample (e.g., benign prostate tissue or cells) to determine whether or not a mammal has prostate cancer. As described herein, the presence of one or more expression levels within a benign prostate sample can indicate that a male mammal (e.g., a male human) has prostate cancer. For example, the presence of an elevated level of SOX-4 expression, an elevated level of CCNB1 expression, an elevated level of GPR37 expression, a reduced level of NACA expression, a reduced level of NR2C2 expression, and/or a reduced level of DLG5 expression within a benign prostate sample obtained from a male human can indicate that that male human has prostate cancer. In some cases, the absence of an elevated level of SOX-4 expression, an elevated level of CCNB1 expression, an elevated level of GPR37 expression, a reduced level of NACA expression, a reduced level of NR2C2 expression, and/or a reduced level of DLG5 expression within a benign prostate sample obtained from a male human can indicate that that male human does not have prostate cancer.

Having the ability to identify male mammals as having prostate cancer as described herein can allow prostate cancer patients to be properly identified and treated in an effective and reliable manner.

In general, one aspect of this document features a method for identifying a mammal as having prostate cancer. The method comprises, or consists essentially of, determining whether or not a benign prostate sample from the mammal contains an elevated level of GPR37 expression, wherein the presence of the elevated level indicates that the mammal has prostate cancer, and wherein the absence of the elevated level indicates that the mammal does not have prostate cancer. The mammal can be a human. The elevated level can be determined using PCR. The elevated level can be determined using immunohistochemistry.

In another aspect, this document features a method for identifying a mammal as having prostate cancer. The method comprises, or consists essentially of, (a) determining whether or not a benign prostate sample from the mammal contains an elevated level of GPR37 expression, (b) classifying the mammal as having prostate cancer if the sample contains the elevated level, and (c) classifying the mammal as not having prostate cancer if the sample lacks the elevated level. The mammal can be a human. The elevated level can be determined using PCR. The elevated level can be determined using immunohistochemistry.

In another aspect, this document features a method for identifying a mammal as having prostate cancer, wherein the method comprises, or consists essentially of, (a) detecting the presence of an elevated level of GPR37 expression in a benign prostate sample from the mammal, and (b) classifying the mammal as having prostate cancer based at least in part on the presence. The mammal can be a human. The elevated level can be detecting using PCR. The elevated level can be detecting using immunohistochemistry.

In another aspect, this document features a method for identifying a mammal as having prostate cancer, wherein the method comprises, or consists essentially of, determining whether or not a benign prostate sample from the mammal contains a reduced level of NACA expression, wherein the presence of the reduced level indicates that the mammal has prostate cancer, and wherein the absence of the reduced level indicates that the mammal does not have prostate cancer. The mammal can be a human. The reduced level can be determined using PCR. The reduced level can be determined using immunohistochemistry.

In another aspect, this document features a method for identifying a mammal as having prostate cancer, wherein the method comprises, or consists essentially of, (a) determining whether or not a benign prostate sample from the mammal contains a reduced level of NACA expression, (b) classifying the mammal as having prostate cancer if the sample contains the reduced level, and (c) classifying the mammal as not having prostate cancer if the sample lacks the reduced level. The mammal can be a human. The reduced level can be determined using PCR. The reduced level can be determined using immunohistochemistry.

In another aspect, this document features a method for identifying a mammal as having prostate cancer, wherein the method comprises, or consists essentially of, (a) detecting the presence of a reduced level of NACA expression in a benign prostate sample from the mammal, and (b) classifying the mammal as having prostate cancer based at least in part on the presence. The mammal can be a human. The reduced level can be detecting using PCR. The reduced level can be detecting using immunohistochemistry.

In another aspect, this document features a method for identifying a mammal as having prostate cancer, wherein the method comprises, or consists essentially of, determining whether or not a benign prostate sample from the mammal contains at least two levels selected from the group consisting of an elevated level of SOX-4 expression, an elevated level of CCNB1 expression, an elevated level of GPR37 expression, a reduced level of NACA expression, a reduced level of NR2C2 expression, and a reduced level of DLG5 expression, wherein the presence of the at least two levels indicates that the mammal has prostate cancer, and wherein the absence of the at least two levels indicates that the mammal does not have prostate cancer. The mammal can be a human. The at least two levels can be determined using PCR. The at least two levels can be determined using immunohistochemistry. The at least two levels can be an elevated level of SOX-4 expression and an elevated level of CCNB1 expression, an elevated level of SOX-4 expression and an elevated level of GPR37 expression, an elevated level of SOX-4 expression and a reduced level of NACA expression, an elevated level of SOX-4 expression and a reduced level of NR2C2 expression, or an elevated level of SOX-4 expression and a reduced level of DLG5 expression. The at least two levels can be an elevated level of CCNB1 expression and an elevated level of GPR37 expression, an elevated level of CCNB1 expression and a reduced level of NACA expression, an elevated level of CCNB1 expression and a reduced level of NR2C2 expression, an elevated level of CCNB1 expression and a reduced level of DLG5 expression, an elevated level of GPR37 expression and a reduced level of NACA expression, an elevated level of GPR37 expression and a reduced level of NR2C2 expression, an elevated level of GPR37 expression and a reduced level of DLG5 expression, a reduced level of NACA expression and a reduced level of NR2C2 expression, a reduced level of NACA expression and a reduced level of DLG5 expression, or a reduced level of NR2C2 expression and a reduced level of DLG5 expression.

In another aspect, this document features a method for identifying a mammal as having prostate cancer, wherein the method comprises, or consists essentially of, (a) determining whether or not a benign prostate sample from the mammal contains at least two levels selected from the group consisting of an elevated level of SOX-4 expression, an elevated level of CCNB1 expression, an elevated level of GPR37 expression, a reduced level of NACA expression, a reduced level of NR2C2 expression, and a reduced level of DLG5 expression, (b) classifying the mammal as having prostate cancer if the sample contains the at least two levels, and (c) classifying the mammal as not having prostate cancer if the sample lacks the at least two levels. The mammal can be a human. The at least two levels can be determined using PCR. The at least two levels can be determined using immunohistochemistry. The at least two levels can be an elevated level of SOX-4 expression and an elevated level of CCNB1 expression, an elevated level of SOX-4 expression and an elevated level of GPR37 expression, an elevated level of SOX-4 expression and a reduced level of NACA expression, an elevated level of SOX-4 expression and a reduced level of NR2C2 expression, or an elevated level of SOX-4 expression and a reduced level of DLG5 expression. The at least two levels can be an elevated level of CCNB1 expression and an elevated level of GPR37 expression, an elevated level of CCNB1 expression and a reduced level of NACA expression, an elevated level of CCNB1 expression and a reduced level of NR2C2 expression, an elevated level of CCNB1 expression and a reduced level of DLG5 expression, an elevated level of GPR37 expression and a reduced level of NACA expression, an elevated level of GPR37 expression and a reduced level of NR2C2 expression, an elevated level of GPR37 expression and a reduced level of DLG5 expression, a reduced level of NACA expression and a reduced level of NR2C2 expression, a reduced level of NACA expression and a reduced level of DLG5 expression, or a reduced level of NR2C2 expression and a reduced level of DLG5 expression.

In another aspect, this document features a method for identifying a mammal as having prostate cancer, wherein the method comprises, or consists essentially of, (a) detecting the presence of at least two levels in a benign prostate sample from the mammal, wherein the at least two levels are selected from the group consisting of an elevated level of SOX-4 expression, an elevated level of CCNB1 expression, an elevated level of GPR37 expression, a reduced level of NACA expression, a reduced level of NR2C2 expression, and a reduced level of DLG5 expression, and (b) classifying the mammal as having prostate cancer based at least in part on the presence. The mammal can be a human. The at least two levels can be detecting using PCR. The at least two levels can be detecting using immunohistochemistry. The at least two levels can be an elevated level of SOX-4 expression and an elevated level of CCNB1 expression, an elevated level of SOX-4 expression and an elevated level of GPR37 expression, an elevated level of SOX-4 expression and a reduced level of NACA expression, an elevated level of SOX-4 expression and a reduced level of NR2C2 expression, or an elevated level of SOX-4 expression and a reduced level of DLG5 expression. The at least two levels can be an elevated level of CCNB1 expression and an elevated level of GPR37 expression, an elevated level of CCNB 1 expression and a reduced level of NACA expression, an elevated level of CCNB 1 expression and a reduced level of NR2C2 expression, an elevated level of CCNB1 expression and a reduced level of DLG5 expression, an elevated level of GPR37 expression and a reduced level of NACA expression, an elevated level of GPR37 expression and a reduced level of NR2C2 expression, an elevated level of GPR37 expression and a reduced level of DLG5 expression, a reduced level of NACA expression and a reduced level of NR2C2 expression, a reduced level of NACA expression and a reduced level of DLG5 expression, or a reduced level of NR2C2 expression and a reduced level of DLG5 expression.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. BP and BPC did not show large scale expression differences. (A) Sammon map of the most variable probesets (350) in BP, BPC, and PCa samples separated the benign prostates from the prostate tumors. By contrast, a map of the most variable probesets (350) in BP and BPC samples did not separate these two groups (B), suggesting that there are no large scale expression differences in the benign prostate tissue from patients with and without prostate cancer. Grey circles, black circles, and open circles represent BP, BPC, and PCa samples, respectively. Similar results were obtained by using more or fewer probesets. PCa refers to prostate cancer; BP refers to benign prostate glands from patients free of prostate cancer; and BPC refers to benign prostate glands from patients with prostate cancer.

FIG. 2. Permutation analysis indicated significant overlap between BPC and PCa in over-expressed probesets with highest signal to noise ratio (SNR) compared with BP. The histogram was generated by random shuffling of BPC and BP class labels. The arrow points to the number of overlapping probesets with correct BP and BPC labels.

FIG. 3. Expression changes compared with BP in BPC were smaller than in PCa. Microarray data for three representative genes are depicted. Grey circles, black circles, and open circles represent BP, BPC, and PCa samples, respectively.

FIG. 4. Boxplot (25% and 75% quantiles) of six genes validated by quantitative RT-PCR. Three up-regulated markers (left of the dashed line) and down-regulated markers (right of the dashed line) in BPC compared with BP are shown. Top and bottom panels are results from the confirmation and the validation sets, respectively. White and grey bars are BP and BPC samples, respectively. *, **, and *** denote p<0.05, p<0.01, and p<0.001, respectively.

FIG. 5A: The ROC plot of the logistic regression model in the qRT-PCR validation set. The model included NACA and CCNB 1 and had an AUC of 0.84.

FIG. 5B: ROC plot of the logistic regression model in the external microarray set (GSE17951) of Wang et al. (Cancer Res., 70:6448-6455 (2010)). The model had an AUC of 0.90.

FIG. 6. Quality control of the microarray samples for the epithelial content. Samples with low concentrations of epithelial cells (dots inside a square) based on the expression of PSA (KLK3) in GSE17951 dataset were excluded. Grey and black dots are BP and BPC samples, respectively.

FIG. 7. DEGAS generated network of genes with differential expression in BPC stroma. Down and up regulated genes in our bulk expression dataset are designated by dashes and solid bars, respectively.

### DETAILED DESCRIPTION

This document provides methods and materials related to identifying prostate cancer in mammals. For example, this document provides methods and materials for identifying male mammals (e.g., male humans) as having prostate cancer by determining whether or not a benign prostate sample (e.g., a benign prostate tissue or cell sample) from the mammal contains cells having an elevated level of SOX-4 expression, an elevated level of CCNB1 expression, an elevated level of GPR37 expression, a reduced level of NACA expression, a reduced level of NR2C2 expression, and/or a reduced level of DLG5 expression. As described herein, if a mammal contains a benign prostate sample with an elevated level of SOX-4 expression, an elevated level of CCNB1 expression, an elevated level of GPR37 expression, a reduced level of NACA expression, a reduced level of NR2C2 expression, and/or a reduced level of DLG5 expression, then that mammal can be classified as having prostate cancer. If a mammal contains a benign prostate sample that lacks an elevated level of SOX-4 expression, an elevated level of CCNB1 expression, an elevated level of GPR37 expression, a reduced level of NACA expression, a reduced level of NR2C2 expression, and/or a reduced level of DLG5 expression, then that mammal can be classified as not having prostate cancer.

The term “elevated level” as used herein with respect to a level of expression (e.g., SOX-4, CCNB1, and/or GPR37 expression) refers to any level that is greater than a reference level for that molecule (e.g., a reference level of SOX-4, CCNB1, and/or GPR37 expression). The term “reference level” as used herein with respect to a particular molecule (e.g., a reference level of SOX-4, CCNB1, and/or GPR37 expression) refers to the level of expression that is typically observed with benign prostate tissue or cells from mammals (e.g., male humans) known to be free of prostate cancer. For example, a reference level of SOX-4 expression can be the average level of SOX-4 expression that is present in benign prostate samples obtained from a random sampling of 50 males free of prostate cancer. In some cases, an elevated level of expression (e.g., SOX-4, CCNB1, and/or GPR37 expression) can be a level that is at least 12, 18, or 25 percent greater than a reference level for that molecule (e.g., a reference level of SOX-4, CCNB1, and/or GPR37 expression).

The term “reduced level” as used herein with respect to a level of expression (e.g., NACA, NR2C2, and/or DLG5 expression) refers to any level that is less than a reference level for that molecule (e.g., a reference level of NACA, NR2C2, and/or DLG5 expression). The term “reference level” as used herein with respect to a particular molecule (e.g., a reference level of NACA, NR2C2, and/or DLG5 expression) refers to the level of expression that is typically observed with benign prostate tissue or cells from mammals (e.g., male humans) known to be free of prostate cancer. For example, a reference level of NACA expression can be the average level of NACA expression that is present in benign prostate samples obtained from a random sampling of 50 males free of prostate cancer. In some cases, a reduced level of expression (e.g., NACA, NR2C2, and/or DLG5 expression) can be a level that is at least 25, 32, or 35 percent less than a reference level for that molecule (e.g., a reference level of NACA, NR2C2, and/or DLG5 expression).

It will be appreciated that levels from comparable samples are used when determining whether or not a particular level is an elevated or reduced level. In some cases, a reference level of expression can be a ratio of an expression value of SOX-4, CCNB1, GPR37, NACA, NR2C2, or DLG5 in a benign sample to an expression value of a control nucleic acid or polypeptide in the sample. A control nucleic acid or polypeptide can be any nucleic acid or polypeptide that has a minimal variation in expression level across various samples of the type for which the nucleic acid or polypeptide serves as a control. For example, DUS2L, EIF2B1, STRADA, NUDC, and ACTB nucleic acids or polypeptides can be used as control nucleic acids or polypeptides in prostate samples.

As described herein, the level of SOX-4, CCNB1, GPR37, NACA, NR2C2, and/or DLG5 expression within a benign prostate sample can be used to determine whether or not a particular mammal has prostate cancer. Any appropriate benign prostate sample can be used as described herein to identify mammals having prostate cancer. For example, prostate tissue samples, prostate cell samples, and prostate needle biopsy specimen can be used to determine whether or not a mammal has prostate cancer.

In addition, any appropriate method can be used to obtain a benign prostate sample. For example, a prostate tissue sample can be obtained by a tissue biopsy or following surgical resection. Once obtained, a sample can be processed prior to measuring a level of expression. For example, a prostate tissue sample can be processed to extract RNA from the sample. Once obtained, the RNA can be evaluated to determine the level of an mRNA of interest. In some embodiments, nucleic acids present within a sample can be amplified (e.g., linearly amplified) prior to determining the level of expression (e.g., using array technology). In another example, a prostate tissue sample can be frozen, and sections of the frozen tissue sample can be prepared on glass slides. The frozen tissue sections can be stored (e.g., at −80° C.) prior to analysis, or they can be analyzed immediately (e.g., by immunohistochemistry with an antibody specific for a particular polypeptide of interest).

Any appropriate methods can be used to determine the level of SOX-4, CCNB1, GPR37, NACA, NR2C2, and/or DLG5 expression within a benign prostate sample. For example, quantitative PCR, in situ hybridization, microarray technology, or sequencing can be used to determine whether or not a particular sample contains an elevated level of mRNA expression for a particular nucleic acid, lacks an elevated level of mRNA expression for a particular nucleic acid, contains a reduced level of mRNA expression for a particular nucleic acid, or lacks a reduced level of mRNA expression for a particular nucleic acid. In some cases, the level of expression can be determined using polypeptide detection methods such as immunochemistry techniques. For example, antibodies specific for SOX-4, CCNB1, GPR37, NACA, NR2C2, or DLG5 polypeptides can be used to determine the polypeptide level in a sample. In some cases, polypeptide-based techniques such as ELISAs and immunocytochemistry techniques can be used to determine whether or not a particular sample contains an elevated level of polypeptide expression for a particular nucleic acid, lacks an elevated level of polypeptide expression for a particular nucleic acid, contains a reduced level of polypeptide expression for a particular nucleic acid, or lacks a reduced level of polypeptide expression for a particular nucleic acid.

Examples of a human SOX-4 nucleic acid can have the sequence set forth in GenBank® Accession No. AI989477 (GI No. 5836358), and a human SOX-4 polypeptide can have the sequence set forth in GenBank® Accession No. Q06945 (GI No. 548952).

Examples of a human CCNB1 nucleic acid can have the sequence set forth in GenBank® Accession No. N90191 (GI No. 1443518), and a human CCNB1 polypeptide can have the sequence set forth in GenBank® Accession No. P14635 (GI No. 116176).

Examples of a human GPR37 nucleic acid can have the sequence set forth in GenBank® Accession No. U87460.1 (GI No. 2076881), and a human GPR37 polypeptide can have the sequence set forth in GenBank® Accession No. A4D0Y6 (GI No. 344235573).

Examples of a human NACA nucleic acid can have the sequence set forth in GenBank® Accession No. AI992187 (GI No. 5839092), and a human NACA polypeptide can have the sequence set forth in GenBank® Accession No. EFB28863 (GI No. 281353279).

Examples of a human NR2C2 nucleic acid can have the sequence set forth in GenBank® Accession No. AI571166 (GI No. 4534540).

Examples of a human DLG5 nucleic acid can have the sequence set forth in GenBank® Accession No. BC002915.1 (GI No. 12804124), and a human DLG5 polypeptide can have the sequence set forth in GenBank® Accession No. Q8TDM6 (GI No. 158939323).

Once the level of SOX-4, CCNB1, GPR37, NACA, NR2C2, and/or DLG5 expression within a benign prostate sample from a mammal is determined, the level(s) can be compared to reference level(s) and used to classify the mammal as having or lacking prostate cancer as described herein. In some cases, a combination of levels of SOX-4, CCNB1, GPR37, NACA, NR2C2, or DLG5 expression can be used to identify a mammal as having or lacking prostate cancer. For example, the presence of an elevated level of SOX-4 expression and an elevated level of CCNB1 expression, or an elevated level of SOX-4 expression and an elevated level of GPR37 expression, or an elevated level of SOX-4 expression and a reduced level of NACA expression, or an elevated level of SOX-4 expression and a reduced level of NR2C2 expression, or an elevated level of SOX-4 expression and a reduced level of DLG5 expression, or an elevated level of CCNB1 expression and an elevated level of GPR37 expression, or an elevated level of CCNB1 expression and a reduced level of NACA expression, or an elevated level of CCNB1 expression and a reduced level of NR2C2 expression, or an elevated level of CCNB1 expression and a reduced level of DLG5 expression, or an elevated level of GPR37 expression and a reduced level of NACA expression, or an elevated level of GPR37 expression and a reduced level of NR2C2 expression, or an elevated level of GPR37 expression and a reduced level of DLG5 expression, or a reduced level of NACA expression and a reduced level of NR2C2 expression, or a reduced level of NACA expression and a reduced level of DLG5 expression, or a reduced level of NR2C2 expression and a reduced level of DLG5 expression can be used to classify a mammal as having prostate cancer.

In some cases, the absence of an elevated level of SOX-4 expression and an elevated level of CCNB1 expression, or an elevated level of SOX-4 expression and an elevated level of GPR37 expression, or an elevated level of SOX-4 expression and a reduced level of NACA expression, or an elevated level of SOX-4 expression and a reduced level of NR2C2 expression, or an elevated level of SOX-4 expression and a reduced level of DLG5 expression, or an elevated level of CCNB1 expression and an elevated level of GPR37 expression, or an elevated level of CCNB1 expression and a reduced level of NACA expression, or an elevated level of CCNB1 expression and a reduced level of NR2C2 expression, or an elevated level of CCNB1 expression and a reduced level of DLG5 expression, or an elevated level of GPR37 expression and a reduced level of NACA expression, or an elevated level of GPR37 expression and a reduced level of NR2C2 expression, or an elevated level of GPR37 expression and a reduced level of DLG5 expression, or a reduced level of NACA expression and a reduced level of NR2C2 expression, or a reduced level of NACA expression and a reduced level of DLG5 expression, or a reduced level of NR2C2 expression and a reduced level of DLG5 expression can be used to classify a mammal as not having prostate cancer.

This document also provides methods and materials to assist medical or research professionals in identifying a mammal as having prostate cancer outcome. Medical professionals can be, for example, doctors, nurses, medical laboratory technologists, and pharmacists. Research professionals can be, for example, principle investigators, research technicians, postdoctoral trainees, and graduate students. A professional can be assisted by (a) determining the level of SOX-4, CCNB1, GPR37, NACA, NR2C2, and/or DLG5 expression within a benign prostate sample, and (b) communicating information about that the level(s) to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

The invention will be further described in the following examples, which do not limit the scope of the invention described in the claims.

### EXAMPLES

### Example 1

### Gene Expression Alterations in Prostate Cancer and Histologically Benign Prostate from Patients with Prostate Cancer

### Abbreviations

PCa: prostate cancer; BP: benign prostate glands from patients free of prostate cancer; BPC: benign prostate glands from patients with prostate cancer; AUC: Area under the ROC curve; ROC: receiver operating characteristics.

### Patient Samples

BP samples were resected prostates from cystoprostatectomy specimens removed for bladder cancer that were free of both prostate cancer and bladder cancers. BPC and PCa samples were obtained from the Mayo Clinic Specialized Program of Research Excellence in Prostate Cancer (SPORE) tumor bank. All patients with PCa and BPC underwent radical prostatectomy, and none received preoperative hormonal therapy, chemotherapy, or radiation therapy. PCa samples were from patients with tumor Gleason score (GS) of 7 and higher and were independent of BPC samples. The discovery step by microarray expression profiling included BP (n=28), BPC (n=36), and PCa (n=37).

In the BPC group, there were 16 samples from patients with Gleason score 6 and 20 samples from patients with Gleason score 8 and higher. Quantitative RT-PCR experiments included BP (n=15) and BPC (n=23) of the discovery samples for confirmation and an independent set of BP (n=18) and BPC (n=33) for validation. The BPC validation set included 15 samples from patients with Gleason score 6 and 18 samples from patients with Gleason score 8 and higher. Finally, sixteen independent benign prostates from radical prostatectomy and cystoprostatectomy operations were used in the laser captured microdissection (LCM) expression profiling to identify stroma related genes in a pathway analysis.

### Microarray Expression Profiling of Bulk Samples

Total RNA (1.0-1.2 microgram) from each sample was isolated by standard kits (Qiagen) and used for labeling and hybridization to the Affymetrix U133Plus2 chips (Affymetrix Corp., Santa Clara, Calif.) following standard protocols.

### Statistical Analysis

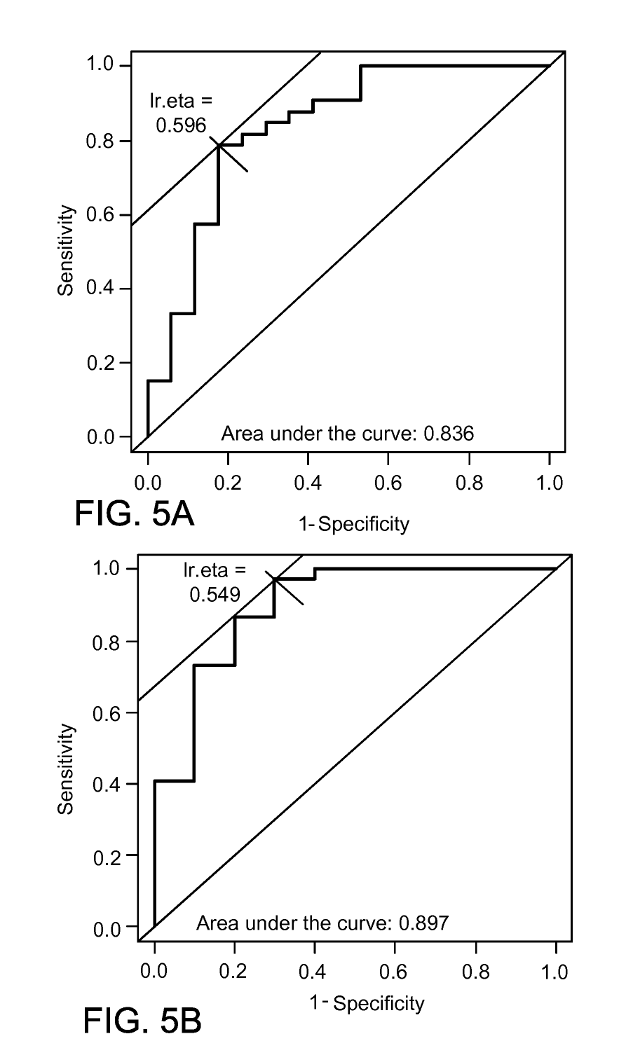

The signal intensity (.cel) files from Mayo samples and from a study described elsewhere (Wang et al., Cancer Res., 70:6448-6455 (2010), GEO record GSE17951) were normalized and processed separately using gcrma package (http at cran.at.r-project.org). Internal and external samples were inspected for the expression of PSA (KLK3) as a quality control for the cellular composition and RNA integrity or non-prostatic tissue sample. Seven samples from GSE17951 set were eliminated because of low PSA expression (FIG. 5).

Two metrics used for gene selection were signal to noise ratio (SNR) and p-values by t-tests. SNR were calculated as SNR=(μ₁−μ₂)/(σ₁+σ₂) where μ's were mean expression values and σ's were maximum of 0.2×μ and standard deviation (Golub et al., Science, 286:531-537 (1999)). It also was required that the average expression in samples over-expressing a gene has greater than 3.5 log₂ intensity. Log₂ expression intensities for the gcrma normalized data ranged from 1 to 16.5. Based on experience with quantitative RT-PCR, gene expression intensities below 3.5 were not reliable and often not detected.

Confirmation and Validation by qRT-PCR

Total RNA (125-500 ng) was used in reverse transcription using Superscript III (Invitrogen). Quantitative PCR was performed in duplicates using PCR arrays from Fluidigm (San Francisco, Calif.). Data were normalized by five genes including ACTB. The other four normalizer genes (DUS2L, EIF2B1, LYK5, and NUDC) were identified based on small standard deviations in BP and BPC samples in the microarray data. Each primer set was tested by a standard curve. Table 1 sets forth the primer sequences. Reported values were calculated as referenceₓ−marker_{g,x}+35, where referenceₓ is the average of the 5 housekeeping gene in sample x, and marker_{g,x} is the raw expression for marker g in sample x.

TABLE 1
Primers used in the quantitative RT-PCR experiments.
Normalizer genes are at the bottom of the table.

| Probeset | Marker | left primer | right primer | amplicon |
|---|---|---|---|---|
| 201291_s_at | TOP2A | agattctggaccaaccttcaac | gcctgcagagttcatctttctt | 83 |
| | | (SEQ ID NO: 1) | (SEQ ID NO: 2) | |
| 204750_s_at | DSC2 | cgcgatcttaatatttgccagt | tttctcggcatctagtttggag | 76 |
| | | (SEQ ID NO: 3) | (SEQ ID NO: 4) | |
| 209631_s_at | GPR37 | aacaaataaatctgacccaacc | atacgccgtgaaatgtccact | 82 |
| | | aa (SEQ ID NO: 5) | (SEQ ID NO: 6) | |
| 204324_s_at | GOLIM4 | tgtgatgttggaaagctcattg | aacaaagaacacctgggaactg | 81 |
| | | (SEQ ID NO: 7) | (SEQ ID NO: 8) | |
| 216867_s_at | PDGFAt | tcgggagaacaaagagacagtg | tactgcttcaccgagtgctaca | 77 |
| | | (SEQ ID NO: 9) | (SEQ ID NO: 10) | |
| 213668_s_at | SOX4 | acttcgagttcccggactactg | caggttggagatgctggactc | 83 |
| | | (SEQ ID NO: 11) | (SEQ ID NO: 12) | |
| 228729_at | CCNB1 | aatggtgaatggacaccaactc | attcttagccaggtgctgcata | 87 |
| | | (SEQ ID NO: 13) | (SEQ ID NO: 14) | |
| 209426_s_at | AMACR | cacgtgaaacagagtgattggt | tggaatgtgcttagagggagat | 75 |
| | | (SEQ ID NO: 15) | (SEQ ID NO: 16) | |
| 210469_at | DLG5 | gagaagcccgcactttctacat | tgcaatctgaacacctgacttg | 76 |
| | | (SEQ ID NO: 17) | (SEQ ID NO: 18) | |
| 222018_at | NACA | cctttgttccttgactccctct | tggaatgaggttccttaattgg | 91 |
| | | (SEQ ID NO: 19) | (SEQ ID NO: 20) | |
| 226848_at | NR2C2 | cagatgtgttcccttcactcttg | cctctgttgatgaatttccaggt | 84 |
| | | (SEQ ID NO: 21) | (SEQ ID NO: 22) | |
| 227751_at | PDCD5 | gggagaaaggctgaatctgttg | aaagggtgggaatggagtca | 70 |
| | | (SEQ ID NO: 23) | (SEQ ID NO: 24) | |
| 1556026_at | IDS2 | ggtggatttgagaagatgtgga | acactacagcattcagggttcc | 84 |
| | | (SEQ ID NO: 25) | (SEQ ID NO: 26) | |

Genes for Normalization

| | | | | |
|---|---|---|---|---|
| 47105_at | DUS2L | ccatcagcctagagcatggac | cctgtcactcagatccaccaa | 74 |
| | | (SEQ ID NO: 27) | (SEQ ID NO: 28) | |
| 201632_at | EIF2B1 | gaatctgcatctggcttcca | ccttcctcgtgttctcttggta | 87 |
| | | (SEQ ID NO: 29) | (SEQ ID NO: 30) | |
| 52169_at | STRADA | gcgaccagcctcattctattta | ggcagcttactacttgcccttt | 82 |
| | | (SEQ ID NO: 31) | (SEQ ID NO: 32) | |
| 210574_s_at | NUDC | agatgatgtatgaccagcgaca | aatctcctgtttcttctgttcgt | 71 |
| | | (SEQ ID NO: 33) | c (SEQ ID NO: 34) | |
| 213867_x_at | ACTB | tcctctcccaagtccacaca | gcacgaaggctcatcattca | 129 |
| | | (SEQ ID NO: 35) | (SEQ ID NO: 36) | |

### Results

### Evidence of Cancer in the Most Up-Regulated Markers in the Benign Prostate Glands of PCa Patients

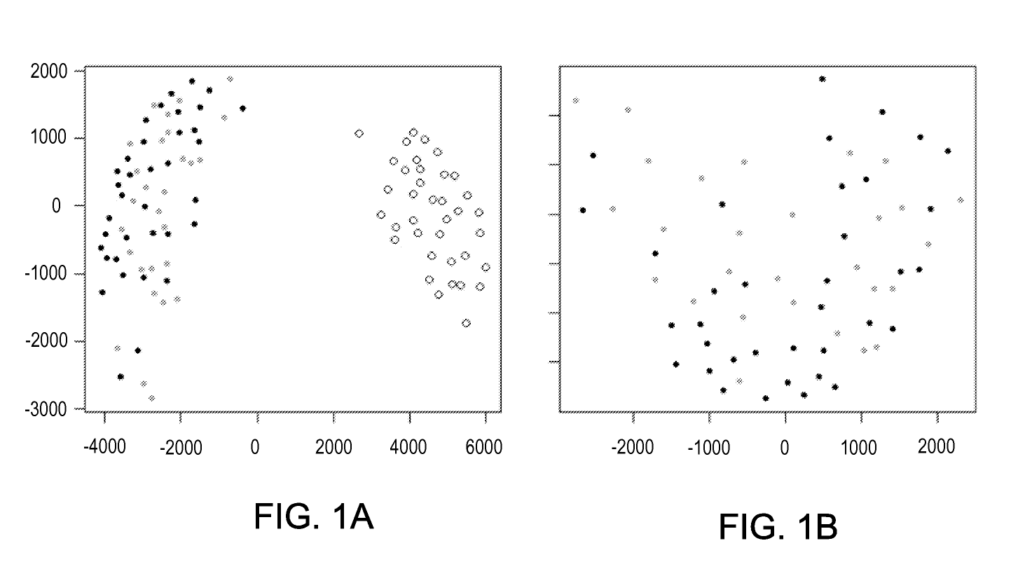

The following was performed to investigate if there were large scale differences in the expression profiles of benign glands from PCa patients (BPC) compared with benign glands from men without PCa (BP). A non-supervised strategy that used most variable probesets (genes) across the samples to generate a Sammon map was adopted. Such a strategy clearly separated prostate cancer from benign prostate tissue (both BP and BPC) on the map (FIG. 1A, left panel), suggesting large scale differences in the expression profiles of tumor and the benign samples. In contrast, mapping of the benign samples did not separate BP from BPC (FIG. 1A, right panel). The two groups were intermixed and did not separate into discrete groups. Changing the number of selected probesets did not alter the patterns. Therefore, it was concluded that the BP and BPC had similar expression profiles.

Gene expression alterations that overlapped between BPC and PCa were then examined. Signal to noise ratio (SNR), a metric designed to identify genes with higher changes between two groups than within two groups (Golub et al., Science, 286:531-537 (1999)), was used to select genes. BPC and PCa were each compared with BP, and genes were ranked by SNR. Within the top 270-285 genes (350 probesets) in the two comparisons, there were 21 overlapping probesets with a significant (p<0.05) increase in both BPC and PCa compared with BP (Table 2). To determine the significance of this overlap, 1000 permutations of BP and BPC sample labels were performed, and the number of overlapping probesets using the same criteria was recorded each time. FIG. 1B is a histogram of the number of overlapping probesets found. The median and the mean number of overlapping probesets were 3 and 5 probesets, respectively. Interestingly, an overlap of 21 probesets were observed in the top 97th percentile, indicating that expression differences between BP and BPC were most likely related to their categorization.

TABLE 2
Overlap in genes and ESTs with 350 highest SNR and significant over-
expression (p < 0.05) in BPC and PCa compared with BP.

| probeset | symbol | p-BPC* | q-BPC** | SNR-BPC^{†} | p - PCa* | non-exonic^{‡} |
|---|---|---|---|---|---|---|
| 201291_s_at | TOP2A | 6.00E−05 | 0.008 | 0.516 | <1.0E−5 | 0 |
| 225767_at | NA | 3.00E−05 | 0.006 | 0.513 | <1.0E−5 | 1 |
| 204750_s_at | DSC2 | 0.00139 | 0.054 | 0.394 | <1.0E−5 | 0 |
| 243648_at | NA | 0.00028 | 0.022 | 0.378 | <1.0E−5 | 1 |
| 209631_s_at | GPR37 | 5.00E−05 | 0.007 | 0.37 | <1.0E−5 | 0 |
| 204324_s_at | GOLIM4 | 9.00E−05 | 0.01 | 0.364 | <1.0E−5 | 0 |
| 238936_at | NA | 0.00071 | 0.037 | 0.36 | <1.0E−5 | 0 |
| 216867_s_at | PDGFA | 0.00078 | 0.04 | 0.339 | <1.0E−5 | 0 |
| 201292_at | TOP2A | 0.00509 | 0.114 | 0.315 | <1.0E−5 | 0 |
| 213668_s_at | SOX4 | <1.00E−05 | 0.002 | 0.315 | <1.0E−5 | 0 |
| 241455_at | NA | 0.00053 | 0.032 | 0.308 | <1.0E−5 | 1 |
| 228729_at | CCNB1 | <1.00E−05 | 0.001 | 0.294 | <1.0E−5 | 1 |
| 243995_at | PTAR1 | 0.00026 | 0.021 | 0.29 | <1.0E−5 | 0 |
| 225762_x_at | LOC284801 | 1.00E−05 | 0.003 | 0.289 | <1.0E−5 | 1 |
| 209426_s_at | AMACR | 0.00168 | 0.06 | 0.286 | <1.0E−5 | 0 |
| 214710_s_at | CCNB1 | <1.00E−05 | 0.001 | 0.28 | <1.0E−5 | 0 |
| 236037_at | LOC202451 | 0.00032 | 0.024 | 0.275 | <1.0E−5 | 0 |
| 204713_s_at | F5 | 0.00115 | 0.048 | 0.271 | <1.0E−5 | 0 |
| 242911_at | MED13L | 0.00042 | 0.028 | 0.269 | <1.0E−5 | 0 |
| 202549_at | VAPB | 0.00013 | 0.013 | 0.269 | <1.0E−5 | 0 |
| 204973_at | GJB1 | 0.00326 | 0.088 | 0.267 | <1.0E−5 | 0 |

Markers selected for validation are shown in bold.
*p-BPC and p-PCa are t-test p-values with the Null hypothesis that the expression in BPC and PCa is not higher than in BP, respectively.
**q-BPC are the q-values calculated from p-BPC
^{†}SNR-BPC are signal to noise ratios of BPC comparisons with BP.
^{‡}non-exonic indicates if the probeset target sequence contains non-exonic regions not present in the RefSeq database.

It is noted that the BPC and PCa were from independent samples and not matched normal-tumor pairs. Therefore, the significant overlaps in the top over-expressed genes between the two categories cannot be attributed to a common background between tumor and adjacent benign prostate tissue samples. Also, it is noteworthy that based on collection of the tissue and review of the H&E slides, collection of BPC in the majority of cases was at a significant distance from tumor, ranging from 1 to 2 cm. However, the possibility that some tumor may have been close to the collected tissue within the three dimensional space of the prostate gland cannot be entirely excluded.

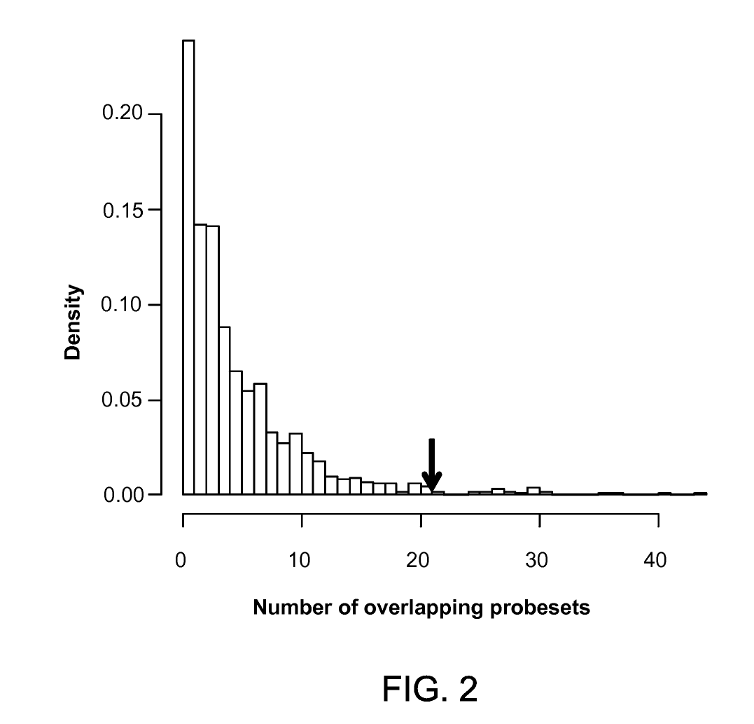

Expression changes in BPC were generally smaller in magnitude than in PCa as shown in FIG. 2 for SOX4, CCNB1, and GPR37. The subtle but detectable expression changes were manifestations of the tumor field effect in the benign prostate glands. From the list of up-regulated candidates, seven genes with the highest SNR (TOP2A, DSC2, GPR37, GOLIM4, PDGFA, SOX4, and CCNB1) were selected for validation by quantitative RT-PCR. AMACR was also included in the validation experiments as altered expression of AMACR by the PCa field effect appears to be described elsewhere (Leav et al., Hum. Pathol., 34:228-233 (2003) and Ananthanarayanan et al., Prostate, 63:341-346 (2005)).

### Down-Regulated Markers in BPC Included a High Percentage of Probesets Containing Non-Exonic Sequences

Fifty of the most down-regulated probesets in BPC compared with BP based on the SNR were selected (Table 3). All probesets were down-regulated in BPC by at least 2-fold, and this down-regulation was statistically significant even after adjusting for multiple comparisons by false discovery rate (FDR) as described elsewhere (Storey and Tibshirani, Proc. Natl. Acad. Sci. USA, 100:9440-9445 (2003); q-values<5×10⁻⁵). More than 70% of the probesets were also down-regulated in PCa compared with BP (p≦0.05). Interestingly, BLAT searches in the RefSeq database revealed that more than 50% of the probesets (26 of 50) had target sequences containing non-exonic sequences. In contrast, the up-regulated list (Table 2) contained less than 25% of such probesets. The significance of transcribed non-exonic sequences could be that they represent long non-coding RNA important for cancer initiation or progression (see, e.g., Gibb et al., Mol. Cancer, 10:38 (2011)) or new gene variants. Five of these probesets that were concomitantly significantly down-regulated in BPC and PCa were selected for validation by quantitative RT-PCR. These included the IDS2 pseudogene and probesets corresponding to NACA, NR2C2, PDCD5, and DLG5 loci.

TABLE 3
Probesets with 50 lowest SNR in BPC compared with BP.

| probeset | symbol | p-BPC* | q-BPC** | SNR-BPC^{†} | p - PCa* | non-exonic^{‡} |
|---|---|---|---|---|---|---|
| 1556026_at | IDS2 | <1.0E−5 | <1.00E−05 | −1.213 | 0.000 | 1 |
| 226670_s_at | PABPC1L | <1.0E−5 | <1.00E−05 | −1.008 | 0.011 | 0 |
| 228331_at | C11orf31 | <1.0E−5 | <1.00E−05 | −0.996 | 1.000 | 1 |
| 219173_at | NA | <1.0E−5 | <1.00E−05 | −0.982 | 0.000 | 0 |
| 236314_at | NA | <1.0E−5 | <1.00E−05 | −0.963 | 0.000 | 0 |
| 204537_s_at | GABRE | <1.0E−5 | <1.00E−05 | −0.957 | 0.000 | 0 |
| 226791_at | KIFC2 | <1.0E−5 | <1.00E−05 | −0.903 | 0.983 | 0 |
| 208498_s_at | AMY1A | <1.0E−5 | <1.00E−05 | −0.897 | 0.003 | 0 |
| 227751_at | PDCD5 | <1.0E−5 | <1.00E−05 | −0.897 | 0.000 | 1 |
| 225191_at | CIRBP | <1.0E−5 | <1.00E−05 | −0.893 | 0.511 | 0 |
| 238540_at | LOC401320 | <1.0E−5 | <1.00E−05 | −0.892 | 0.000 | 0 |
| 213046_at | PABPN1 | <1.0E−5 | <1.00E−05 | −0.885 | 0.609 | 1 |
| 219775_s_at | CPLX3 | <1.0E−5 | 1.00E−05 | −0.884 | 0.000 | 0 |
| 203146_s_at | GABBR1 | <1.0E−5 | <1.00E−05 | −0.87 | 0.001 | 0 |
| 236518_at | C9orf86 | <1.0E−5 | <1.00E−05 | −0.87 | 0.103 | 1 |
| 1555870_at | RNF207 | <1.0E−5 | <1.00E−05 | −0.857 | 0.062 | 1 |
| 1552327_at | ARMCX4 | <1.0E−5 | <1.00E−05 | −0.855 | 0.000 | 0 |
| 210424_s_at | GOLGA8A | <1.0E−5 | <1.00E−05 | −0.853 | 0.000 | 0 |
| 1559096_x_at | FBX09 | <1.0E−5 | <1.00E−05 | −0.848 | 0.000 | 1 |
| 221973_at | NA | <1.0E−5 | <1.00E−05 | −0.847 | 1.000 | 0 |
| 1555858_at | LOC440944 | <1.0E−5 | <1.00E−05 | −0.842 | 0.000 | 1 |
| 210425_x_at | GOLGA8B | <1.0E−5 | <1.00E−05 | −0.832 | 0.000 | 0 |
| 1555938_x_at | VIM | <1.0E−5 | <1.00E−05 | −0.832 | 0.000 | 1 |
| 220954_s_at | PILRB | <1.0E−5 | <1.00E−05 | −0.825 | 0.014 | 0 |
| 236832_at | LOC221442 | <1.0E−5 | <1.00E−05 | −0.821 | 0.002 | 0 |
| 214163_at | C1orf41 | <1.0E−5 | <1.00E−05 | −0.815 | 0.000 | 1 |
| 238456_at | NA | <1.0E−5 | <1.00E−05 | −0.807 | 0.000 | 0 |
| 229870_at | LOC644656 | <1.0E−5 | <1.00E−05 | −0.8 | 1.000 | 0 |
| 210469_at | DLG5 | <1.0E−5 | 3.00E−05 | −0.797 | 0.000 | 1 |
| 244677_at | PER1 | <1.0E−5 | <1.00E−05 | −0.796 | 0.000 | 1 |
| 241672_at | LOC400120 | <1.0E−5 | 4.00E−05 | −0.793 | 0.000 | 0 |
| 1553292_s_at | FLJ25006 | <1.0E−5 | <1.00E−05 | −0.793 | 0.769 | 1 |
| 228506_at | NSMCE4A | <1.0E−5 | <1.00E−05 | −0.791 | 0.000 | 1 |
| 222018_at | NACA | <1.0E−5 | <1.00E−05 | −0.785 | 0.050 | 1 |
| 232291_at | MIHG1 | <1.0E−5 | <1.00E−05 | −0.777 | 0.003 | 0 |
| 212913_at | MSH5 | <1.0E−5 | <1.00E−05 | −0.777 | 0.988 | 1 |
| 232262_at | PIGL | <1.0E−5 | <1.00E−05 | −0.77 | 0.000 | 1 |
| 1558938_at | C14orf122 | <1.0E−5 | <1.00E−05 | −0.769 | 0.438 | 1 |
| 222927_s_at | CPLX3 | <1.0E−5 | 1.00E−05 | −0.767 | 0.000 | 0 |
| 1559094_at | FBX09 | <1.0E−5 | <1.00E−05 | −0.767 | 0.025 | 1 |
| 217538_at | SGSM2 | <1.0E−5 | <1.00E−05 | −0.764 | 0.004 | 1 |
| 228465_at | NA | <1.0E−5 | <1.00E−05 | −0.758 | 0.003 | 0 |
| 226848_at | NR2C2 | <1.0E−5 | <1.00E−05 | −0.758 | 0.003 | 1 |
| 213703_at | LOC150759 | <1.0E−5 | <1.00E−05 | −0.758 | 1.000 | 1 |
| 228528_at | NA | <1.0E−5 | <1.00E−05 | −0.747 | 0.000 | 1 |
| 1555860_x_at | LOC440944 | <1.0E−5 | <1.00E−05 | −0.745 | 0.000 | 1 |
| 241755_at | UQCRC2 | <1.0E−5 | <1.00E−05 | −0.745 | 0.000 | 1 |
| 1552774_a_at | SLC25A27 | <1.0E−5 | <1.00E−05 | −0.742 | 0.001 | 0 |
| 228847_at | EXOC3 | <1.0E−5 | <1.00E−05 | −0.738 | 0.993 | 0 |
| 228030_at | RBM6 | <1.0E−5 | <1.00E−05 | −0.738 | 1.000 | 1 |

All 50 probesets were significant (p < 1.0E−5). Markers selected for validation are shown in bold.

### Quantitative RT-PCR Confirmation and Validation of Selected Up- and Down-Regulated Markers in BPC

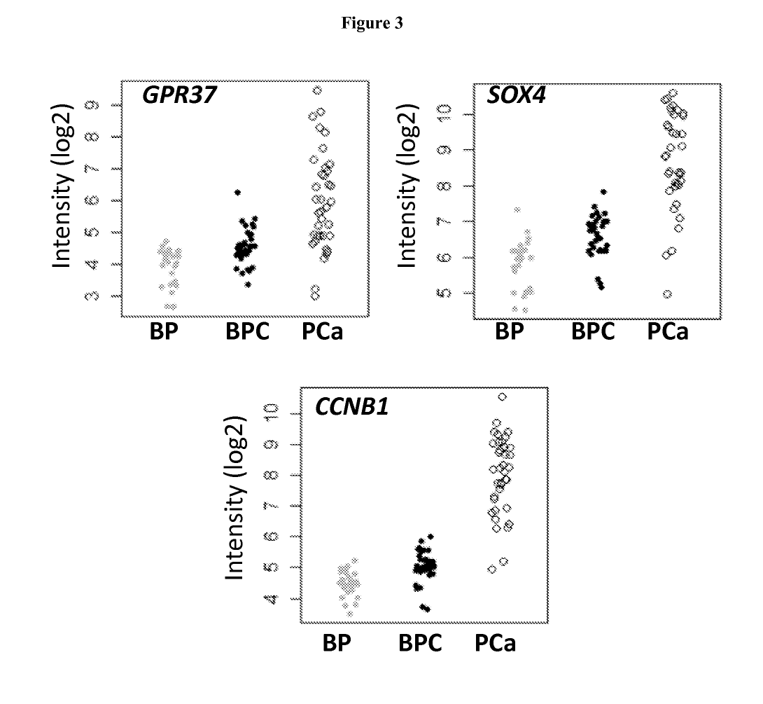

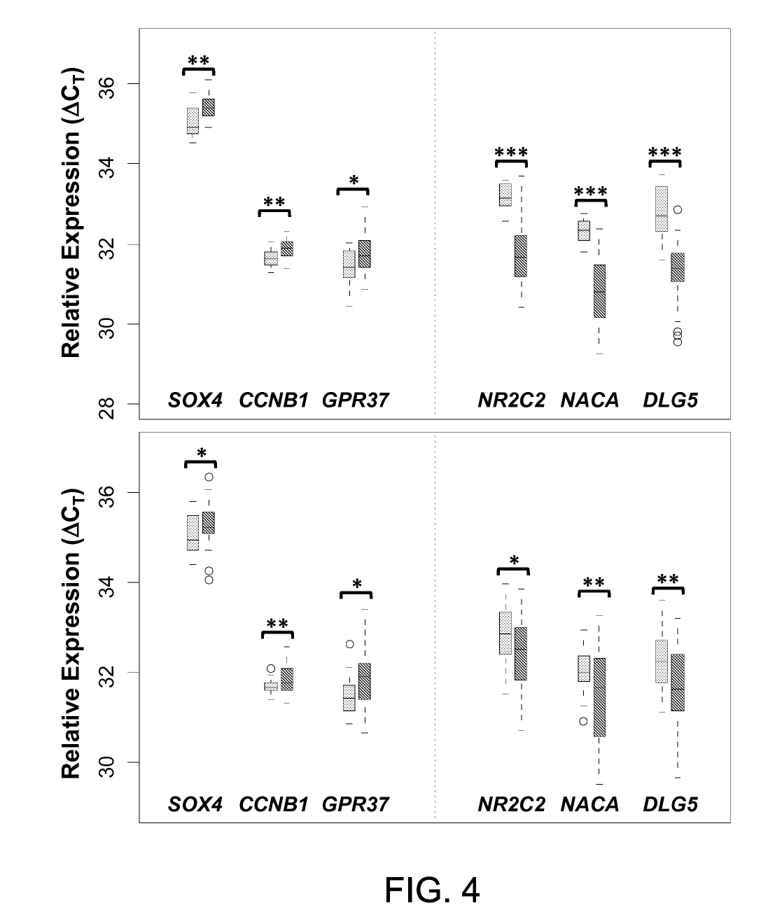

Real time qRT-PCR was used to confirm the findings in the discovery step and to validate in independent samples. Confirmation used a portion of the microarray samples. With the exception of PDGFA, the average expression levels of all markers in BPC and BP agreed with the expected trend based on the microarray data (Table 4). GPR37, SOX4, and CCNB1 were among the up-regulated genes that were confirmed and validated in the independent samples (p≦0.05). GOLIM4 was validated in the independent set (p=0.052) even though the lower expression of this gene in the confirmation set was statistically not significant (p=0.173). DLG5, NACA, and NR2C2 were confirmed and also significantly down-regulated in independent samples, while PDCD5 was marginal (0=0.063). FIG. 3 is a boxplot of three up- and three down-regulated markers in BPC compared with BP.

TABLE 4
Quantitative RT-PCR confirmation and validation data.
Up-regulated Markers

| Marker | p-gr-cnf* | p-gr-val* | Confirmed [symbol] | trend^{¥} |
|---|---|---|---|---|
| GPR37 | 0.018 | 0.03 | y | y |
| SOX4 | 0.003 | 0.03 | y | y |
| CCNB1 | 0.002 | 0.007 | y | y |
| GOLIM4 | 0.173 | 0.052 | n | y |
| TOP2A | 0.018 | 0.267 | y | y |
| AMACR | 0.093 | 0.1 | n | y |
| DSC2 | 0.398 | 0.476 | n | y |
| PDGFA | 0.255 | 0.716 | n | n |

Down-regulated Markers

| Marker | p-ls-cnf** | p-ls-val** | Confirmed [symbol] | trend^{¥} |
|---|---|---|---|---|
| DLG5 | <0.001 | 0.004 | y | y |
| NACA | <0.001 | 0.003 | y | y |
| NR2C2 | <0.001 | 0.02 | y | y |
| PDCD5 | <0.001 | 0.063 | y | y |
| IDS2 | <0.001 | 0.254 | y | y |

Significant values are shown in bold (p ≦ 0.05). PDCD5 was marginal (p = 0.063). All but one marker (PDGFA) had the expected trend based on the microarray data in the confirmation and validation sets.
*p-gr-cnf and p-gr-val are t-test p-values with the Null hypothesis that the expression in BPC is not higher than in BP in the confirmation and validation sets, respectively.
**p-ls-cnf and p-ls-val are t-test p-values with the Null hypothesis that the expression in BPC is not less than in BP in the confirmation and validation sets, respectively. [symbol]
Confirmed indicates if the selected marker was significant in the confirmation set.
^{¥}trend indicates if the BPC average expression was higher than BP for the up-regulated markers and lower than BP for the down-regulated markers.

A Statistical Model Based on Two Markers Stratified BPC from BP

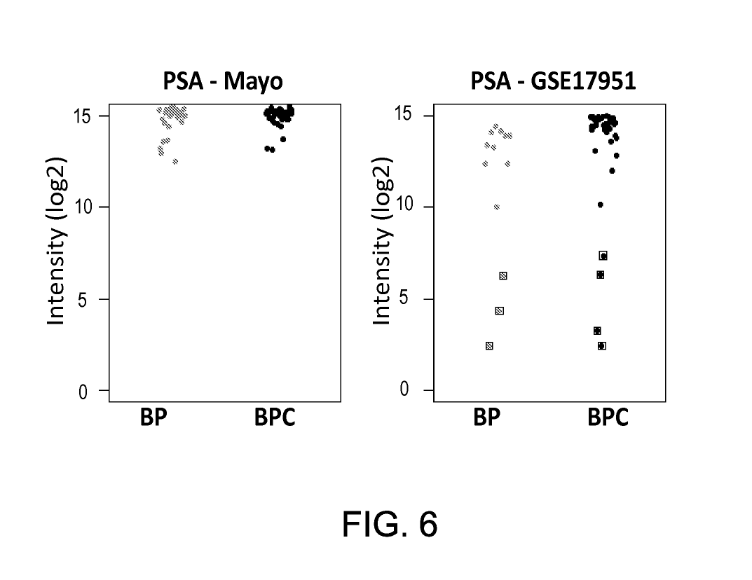

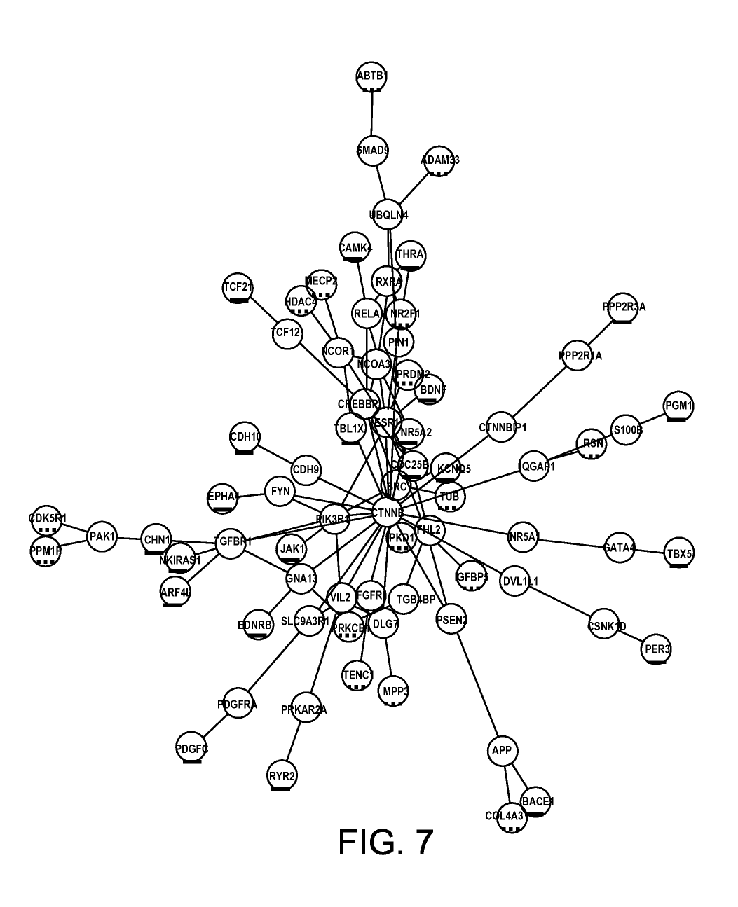

The ability of the markers to distinguish BPC from BP in the qRT-PCR data was examined by a logistic regression that included two markers. Both in the confirmation and in the validation sets, a model that included NACA and CCNB1 produced the maximum area under the curve (AUC) in the ROC plot. FIG. 5A is the ROC plot in the qRT-PCR validation set with an AUC of 0.84. The predictive ability of this model also was examined in the public microarray dataset by Wang et al. (GSE17951). Since that study was focused on a stroma signature, samples with little epithelial content based on PSA expression were first eliminated (FIG. 6). Model coefficients were computed in the microarray data and applied to the Wang et al. dataset. FIG. 5B is the ROC plot for the model with the Wang et al. dataset with an AUC of 0.90. The AUC of the model that included all samples in the Wang et al. database was 0.89. These results indicate that the field effect markers can discriminate between BP and BPC with high accuracy.

### Enriched Gene Ontology (GO) Categories in the BPC Stroma

The following was performed to investigate if the microarray data from bulk samples could detect dysregulated pathways in prostate stroma at large distances from prostate tumors. Genes with prominent expression in stroma were first identified by analyzing expression data from pure stroma and epithelial cell populations collected by laser capture microdissection (LCM). Among the identified genes, there were 218 genes that also were differentially expressed between BPC and BP in the bulk microarray data. Most of the differential expression appeared to occur in the stroma as the expression levels of these genes in stroma were on the average approximately 5 fold higher than the epithelia. These genes were examined by the MATISSE software package as described elsewhere (Ulitsky and Shamir, BMC Syst. Biol., 1:8 (2007)). The DEGAS module in the package identified a network of 91 genes that included 47 of 218 (˜22%) genes in the list (FIG. 6). Enriched GO categories in the network were examined by the TANGO module for multiple comparison correction. Three GO categories remained significant (Table 5). PDGFR signaling was one of the notable carcinogenesis related categories on the list. Also, the enrichment of the “regulation of epithelial cell differentiation” category in BPC stroma was noted. Even though this analysis was limited by the number of genes, it was possible to identify the cancer fingerprint in the enriched GO categories in BPC stroma.

TABLE 5
GO categories enriched by the network identified by DEGAS.

| Enriched GO category | p-value* |
|---|---|
| PDGFR signaling | 0.028 |
| regulation of epithelial cell differentiation | 0.017 |
| peptidyl-tyrosine phosphorylation | 0.014 |

*p value corrected for multiple comparison by TANGO

The results provided herein identify a common cancer transcriptome between histologically benign prostate tissue adjacent to PCa and prostate cancer. A logistic regression model, which that included a down-regulated marker and an up-regulated marker in BPC compared with BP, predicted the presence of tumor in the prostate gland with high accuracy in independent quantitative RT-PCR samples (AUC=0.84) and in an external microarray dataset from Wang et al. (AUC=0.90). These findings provide strong evidence that the transcriptome profiles of the benign prostatic tissue provided herein can indicate the presence or absence of PCa. The results provided herein also demonstrate that independent PCa expression profiles used to guide the selection process allowed for a greater likelihood of identifying markers that would validate.

In addition, the results provided herein focused on transcriptomic alterations that occurred in morphologically benign prostate tissue in prostate glands that harbor cancer. The presence of a field effect was confirmed, and it was found to occur at some distance from the prostate tumors. The results provided herein also validated field effect biomarkers that can be used as valuable tools to choose the correct intervention strategy or in clinical assays aimed at identifying men who potentially have prostate cancer but whose prostate needle biopsy specimen is negative.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-42. (canceled)

43. A method for identifying a male human as having prostate cancer, wherein said method comprises:
(a) performing a real time quantitative reverse transcription polymerase chain reaction using a benign prostate sample from said male human and primers designed to amplify NACA nucleic acid, under conditions wherein a raw expression value for NACA mRNA is obtained,
(b) performing a real time quantitative reverse transcription polymerase chain reaction using a benign prostate sample from said male human and primers designed to amplify CCNB1 nucleic acid, under conditions wherein a raw expression value for CCNB1 mRNA is obtained,
(c) performing a real time quantitative reverse transcription polymerase chain reaction using a benign prostate sample from said male human and primers designed to amplify a normalizer gene nucleic acid, under conditions wherein a measured expression value for mRNA of said normalizer gene is obtained,
(d) obtaining a final expression value for said NACA mRNA by normalizing said raw expression value for NACA mRNA using said measured expression value for mRNA of said normalizer gene,
(e) obtaining a final expression value for said CCNB1 mRNA by normalizing said raw expression value for CCNB1 mRNA using said measured expression value for mRNA of said normalizer gene,
(f) comparing said final expression value for said NACA mRNA to a reference level of NACA mRNA to determine that said final expression value for said NACA mRNA is reduced as compared to said reference level of NACA mRNA, wherein said reference level of NACA mRNA is a value of NACA mRNA expression that is observed within benign prostate tissue from a control male human known to be free of prostate cancer and that is normalized to the level of expression of said normalizer gene within said benign prostate tissue,
(g) comparing said final expression value for said CCNB1 mRNA to a reference level of CCNB1 mRNA to determine that said final expression value for said CCNB1 mRNA is elevated as compared to said reference level of CCNB1 mRNA, wherein said reference level of CCNB1 mRNA is a value of CCNB1 mRNA expression that is observed within benign prostate tissue from a control male human known to be free of prostate cancer and that is normalized to the level of expression of said normalizer gene within said benign prostate tissue, and
(h) classifying said male human as having prostate cancer based at least in part on the comparison of step (f) and the comparison of step (g).

44. The method of claim 43, wherein one of said primers designed to amplify NACA nucleic acid comprises the sequence set forth in SEQ ID NO:19, and another of said primers designed to amplify NACA nucleic acid comprises the sequence set forth in SEQ ID NO:20.

45. The method of claim 43, wherein one of said primers designed to amplify CCNB1 nucleic acid comprises the sequence set forth in SEQ ID NO:13, and another of said primers designed to amplify CCNB1 nucleic acid comprises the sequence set forth in SEQ ID NO:14.

46. The method of claim 43, wherein normalizer gene is selected from the group consisting of DUS2L, EIF2B1, STRADA, NUDC, and ACTB.

47. A method for identifying a male human as having prostate cancer, wherein said method comprises:
(a) performing a real time quantitative reverse transcription polymerase chain reaction using a benign prostate sample from said male human and primers designed to amplify NACA nucleic acid, under conditions wherein a raw expression value for NACA mRNA is obtained,
(b) performing a real time quantitative reverse transcription polymerase chain reaction using a benign prostate sample from said male human and primers designed to amplify CCNB1 nucleic acid, under conditions wherein a raw expression value for CCNB1 mRNA is obtained,
(c) performing multiple real time quantitative reverse transcription polymerase chain reactions wherein each of said multiple real time quantitative reverse transcription polymerase chain reactions comprises using a benign prostate sample from said male human and primers designed to amplify a different normalizer gene nucleic acid, under conditions wherein a measured expression value for mRNA of each different normalizer gene is obtained,
(d) calculating an average normalization value using said measured expression value for mRNA of each different normalizer gene,
(e) obtaining a final expression value for said NACA mRNA by normalizing said raw expression value for NACA mRNA using said average normalization value,
(f) obtaining a final expression value for said CCNB1 mRNA by normalizing said raw expression value for CCNB1 mRNA using said average normalization value,
(g) comparing said final expression value for said NACA mRNA to a reference level of NACA mRNA to determine that said final expression value for said NACA mRNA is reduced as compared to said reference level of NACA mRNA, wherein said reference level of NACA mRNA is a value of NACA mRNA expression that is observed within benign prostate tissue from a control male human known to be free of prostate cancer and that is normalized to an average normalization value of expression of each different normalizer gene within said benign prostate tissue,
(h) comparing said final expression value for said CCNB1 mRNA to a reference level of CCNB1 mRNA to determine that said final expression value for said CCNB1 mRNA is elevated as compared to said reference level of CCNB1 mRNA, wherein said reference level of CCNB1 mRNA is a value of CCNB1 mRNA expression that is observed within benign prostate tissue from a control male human known to be free of prostate cancer and that is normalized to an average normalization value of expression of each different normalizer gene within said benign prostate tissue, and
(i) classifying said male human as having prostate cancer based at least in part on the comparison of step (g) and the comparison of step (h).

48. The method of claim 47, wherein one of said primers designed to amplify NACA nucleic acid comprises the sequence set forth in SEQ ID NO:19, and another of said primers designed to amplify NACA nucleic acid comprises the sequence set forth in SEQ ID NO:20.

49. The method of claim 47, wherein one of said primers designed to amplify CCNB1 nucleic acid comprises the sequence set forth in SEQ ID NO:13, and another of said primers designed to amplify CCNB1 nucleic acid comprises the sequence set forth in SEQ ID NO:14.

50. The method of claim 47, wherein said different normalizer genes are selected from the group consisting of DUS2L, EIF2B1, STRADA, NUDC, and ACTB.

51. The method of claim 47, wherein said different normalizer genes are DUS2L, EIF2B1, STRADA, NUDC, and ACTB.


SEQUENCE LISTING (36 sequences)

SEQ ID NO: 1 - DNA, 22 bp, Homo sapiens
agattctggaccaaccttcaac

SEQ ID NO: 2 - DNA, 22 bp, Homo sapiens
gcctgcagagttcatctttctt

SEQ ID NO: 3 - DNA, 22 bp, Homo sapiens
cgcgatcttaatatttgccagt

SEQ ID NO: 4 - DNA, 22 bp, Homo sapiens
tttctcggcatctagtttggag

SEQ ID NO: 5 - DNA, 24 bp, Homo sapiens
aacaaataaatctgacccaaccaa

SEQ ID NO: 6 - DNA, 21 bp, Homo sapiens
atacgccgtgaaatgtccact

SEQ ID NO: 7 - DNA, 22 bp, Homo sapiens
tgtgatgttggaaagctcattg

SEQ ID NO: 8 - DNA, 22 bp, Homo sapiens
aacaaagaacacctgggaactg

SEQ ID NO: 9 - DNA, 22 bp, Homo sapiens
tcgggagaacaaagagacagtg

SEQ ID NO: 10 - DNA, 22 bp, Homo sapiens
tactgcttcaccgagtgctaca

SEQ ID NO: 11 - DNA, 22 bp, Homo sapiens
acttcgagttcccggactactg

SEQ ID NO: 12 - DNA, 21 bp, Homo sapiens
caggttggagatgctggactc

SEQ ID NO: 13 - DNA, 22 bp, Homo sapiens
aatggtgaatggacaccaactc

SEQ ID NO: 14 - DNA, 22 bp, Homo sapiens
attcttagccaggtgctgcata

SEQ ID NO: 15 - DNA, 22 bp, Homo sapiens
cacgtgaaacagagtgattggt

SEQ ID NO: 16 - DNA, 22 bp, Homo sapiens
tggaatgtgcttagagggagat

SEQ ID NO: 17 - DNA, 22 bp, Homo sapiens
gagaagcccgcactttctacat

SEQ ID NO: 18 - DNA, 22 bp, Homo sapiens
tgcaatctgaacacctgacttg

SEQ ID NO: 19 - DNA, 22 bp, Homo sapiens
cctttgttccttgactccctct

SEQ ID NO: 20 - DNA, 22 bp, Homo sapiens
tggaatgaggttccttaattgg

SEQ ID NO: 21 - DNA, 23 bp, Homo sapiens
cagatgtgttcccttcactcttg

SEQ ID NO: 22 - DNA, 23 bp, Homo sapiens
cctctgttgatgaatttccaggt

SEQ ID NO: 23 - DNA, 22 bp, Homo sapiens
gggagaaaggctgaatctgttg

SEQ ID NO: 24 - DNA, 20 bp, Homo sapiens
aaagggtgggaatggagtca

SEQ ID NO: 25 - DNA, 22 bp, Homo sapiens
ggtggatttgagaagatgtgga

SEQ ID NO: 26 - DNA, 22 bp, Homo sapiens
acactacagcattcagggttcc

SEQ ID NO: 27 - DNA, 21 bp, Homo sapiens
ccatcagcctagagcatggac

SEQ ID NO: 28 - DNA, 21 bp, Homo sapiens
cctgtcactcagatccaccaa

SEQ ID NO: 29 - DNA, 20 bp, Homo sapiens
gaatctgcatctggcttcca

SEQ ID NO: 30 - DNA, 22 bp, Homo sapiens
ccttcctcgtgttctcttggta

SEQ ID NO: 31 - DNA, 22 bp, Homo sapiens
gcgaccagcctcattctattta

SEQ ID NO: 32 - DNA, 22 bp, Homo sapiens
ggcagcttactacttgcccttt

SEQ ID NO: 33 - DNA, 22 bp, Homo sapiens
agatgatgtatgaccagcgaca

SEQ ID NO: 34 - DNA, 24 bp, Homo sapiens
aatctcctgtttcttctgttcgtc

SEQ ID NO: 35 - DNA, 20 bp, Homo sapiens
tcctctcccaagtccacaca

SEQ ID NO: 36 - DNA, 20 bp, Homo sapiens
gcacgaaggctcatcattca